(19) United States
(12) Patent Application Publication
(10) Pub. No.: US 2011/0256218 A1
(43) Pub. Date: Oct. 20, 2011

(54) CONTROLLED RELEASE COMPOSITIONS COMPRISING MECLIZINE OR RELATED PIPERAZINE DERIVATIVES

(75) Inventors: Gopi M. VENKATESH, Vandalia, OH (US); Troy M. HARMON, Lansdale, PA (US)

(73) Assignee: EURAND, INC., Vandalia, OH (US)

(21) Appl. No.: 12/984,432

(22) Filed: Jan. 4, 2011

Related U.S. Application Data
(60) Provisional application No. 61/292,084, filed on Jan. 4, 2010.

(51) Int. Cl. A61K 9/58; A61P 1/08; A61K 9/14; A61K 31/495; A61K 9/56; A61K 9/30

(52) U.S. Cl. 424/459; 424/465; 424/490; 424/493; 424/494; 424/497; 427/2.21; 514/255.04

(57) ABSTRACT

The present invention provides pharmaceutically acceptable compositions for once-daily dosing comprising a piperazine derivative of H₁-receptor antagonists, or its salt, and/or solvate and methods of making and using the compositions in the treatment of treating vertigo and other diseases. The present invention also provides once-a-day dosage forms as orally disintegrating tablets comprising compositions of the present invention.

### CROSS REFERENCE TO RELATED APPLICATIONS

The present application claims priority to U.S. Provisional Application No. 61/292,084 filed Jan. 4, 2010, which is incorporated herein by reference in its entirety for all purposes.

### BACKGROUND OF THE INVENTION

Dizziness is a common complaint among patients seen by primary care physicians, neurologists, and otolaryngologists. The most common causes of dizziness are peripheral vestibular disorders, but central nervous system disorders must be excluded. Vertigo, a subtype of dizziness, is an uncomfortable feeling of movement when there is no actual movement. The feeling of motion is commonly described as spinning or whirling but also may include sensation of falling or tilting. Vertigo can cause nausea and vomiting. It may be difficult to maintain balance, walk, or stand. Causes of vertigo include nerve, blood flow, or inner ear problems (severe infection). If vertigo is severe or frequent, treatment will depend on the specific cause. The sudden onset of vertigo usually indicates a peripheral vestibular disorder (inner ear disturbance; e.g., benign paroxysmal positional vertigo (BPPV), Ménière disease, vestibular neuritis). Symptoms of BPPV usually last a few seconds to a few minutes and are intermittent (i.e., come and go). Symptoms of Ménière disease and vestibular neuritis include vertigo, hearing loss, ringing in the ears (tinnitus), and ear pressure that often lasts hours to days. According to the American Academy of Neurology, the most effective treatment for BPPV caused by ear crystals in the posterior semicircular canal, is a technique called the canalith repositioning procedure, or the Epley maneuver. BPPV that does not respond to canalith repositioning may be treated with meclizine (Antivert®), an oral antiemetic that can be taken up to 3 times a day, or only as needed. Meclizine may cause drowsiness, dry mouth, and blurred vision. The recurrence rate of peripheral vestibulopathy is out of 3 after one year and 1 out of 2 after five years.
Dizziness has ˜30% prevalence in the general population, or 90 million in the United States, half of which is vertigo due to peripheral vestibulopathy (inner ear dysfunction).
BPPV is diagnosed in 25% of all cases of dizziness, or 22 million patients.
Vestibular neuronitis and labyrinthitis is prevalent in 15% of all peripheral vestibulopathies, or 6 million patients.
56% (or ˜8 million) of a total of ˜15 million elderly patients in the US experience vertigo due to peripheral vestibulopathy.
Peripheral vertigo in the elderly tends to be more persistent and recurrent.
Sufferers often experience a reduced daily living score and depression.
Patients may be too frail to undergo posture maneuver therapy.

Meclizine is the active ingredient in the brand Antivert® (Pfizer); originally approved in 1957. Meclizine dihydrochloride monohydrate is a member of the piperazine class of H₁-receptor antagonists. It is chemically 1-(p-chloro-α-phenylbenzyl)-4-(m-methylbenzyl)piperazine dihydrochloride monohydrate. Its structure is shown below.

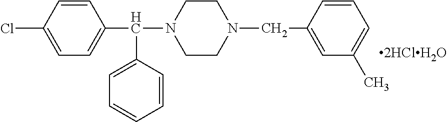

Meclizine 2HCl.H₂O is a weakly basic chemical entity with a pKa of 6.2 and logP of 5.87. It is slightly soluble in 0.1 N HCl, very slightly soluble in water at neutral pH (e.g., 1 mg/mL), but practically insoluble at pH 6.0 or above. It is well absorbed after oral administration. The onset of action of meclizine is about 1 hour, with effects lasting between 8-24 hours. The plasma half-life in humans is about 6 hours. Meclizine is generally used for nausea relief due to motion sickness. It is also used to control the nausea resulting from vestibular disease, a syndrome characterized by vertigo and loss of balance. Common side effects (e.g., sedation reported at 20%-30%, dry mouth, and blurred vision) are problematic for patients taking meclizine for several weeks to treat persistent types of vertigo, particularly in the elderly. Persistent vertigo afflicts multiple patient types, including vestibular neuronitis and labyrinthitis patients, which are the most persistent types of vertigo and often require drug intervention for several weeks (˜6 million patients in the United States of America). Elderly patients are more susceptible to recurrent and persistent patterns of vertigo (˜8 million patients in the United States of America).

Thus there is an unmet medical need to provide a patient compliant dosing regimen with a reduced incidence of side effects. The compositions of the present invention provide improved delivery of poorly soluble, weakly basic piperazine derivatives of H₁-receptor antagonists such as meclizine or pharmaceutically acceptable salt or solvate thereof, with drug release profiles suitable for once-daily dosing regimens, i.e., to maintain therapeutically effective plasma concentrations over 12-20 hours with minimal difference at steady-state between peak and trough levels and an option for bedtime administration.

### SUMMARY OF THE INVENTION

In one embodiment, the present invention is related to a pharmaceutical composition which comprises at least one population of controlled-release (CR) particles, wherein each CR particle comprises a core comprising a pharmaceutically acceptable piperazine derivative of H₁-receptor antagonists, e.g. meclizine, and a polymeric binder, a first coating comprising a water-insoluble polymer alone or a water-insoluble polymer in combination with an optional water-soluble polymer, and a second optional coating disposed over said first coating comprising an enteric polymer and an optional water-insoluble polymer, wherein the first coating is essentially free of enteric polymers.

In another embodiment, the present invention is related to a pharmaceutical composition which comprises at least one population of controlled-release (CR) particles, wherein each CR particle comprises a core comprising a pharmaceutically acceptable organic acid and a polymeric binder, a first coating disposed over said acid core, comprising a water-insoluble polymer alone or a water-insoluble polymer in combination with an optional water-soluble or enteric polymer to produce a CR coated acid core, and a second coating disposed over said CR acid core, comprising a weakly basic, piperazine derivative of H₁-receptor antagonists, such as meclizine, and a polymeric binder, and a third coating disposed over said drug core comprising an enteric polymer and optionally a water-insoluble polymer. The pharmaceutical composition further comprises a second population of IR particles, wherein the IR particle of the second population comprises a weakly basic, piperazine derivative of H₁-receptor antagonists, such as meclizine, or a pharmaceutically acceptable salt, polymorph, isomer, hydrate, solvate, and/or ester thereof. The current invention also provides for a taste-masked component in the form of an orally disintegrating tablet.

In accordance with certain embodiments of the present invention, the controlled release (CR) composition comprises a plurality of meclizine-containing particles, the particle comprising:

(a) a core comprising an organic acid layer comprising a pharmaceutically acceptable organic acid and a polymeric binder disposed over an inert core such as a sugar sphere or a cellulosic sphere;

(b) a first coating disposed over the acid core comprising at least one water-insoluble polymer;

(c) a second coating disposed over the coated acid core comprising meclizine and a polymeric binder; and

(d) a third coating disposed over said second coating comprising a water insoluble polymer optionally in combination with a water-soluble polymer to produce a meclizine SR bead;
wherein said organic acid in the acid core solubilizes the meclizine by creating an acidic pH microenvironment inside the meclizinecoated bead prior to releasing it into the intestinal region where the meclizine would otherwise be practically insoluble.

In another embodiment, the present invention is directed to a pharmaceutical composition comprising controlled-release (CR) beads, wherein said CR beads comprise a solid dispersion of meclizine or a pharmaceutically acceptable salt thereof and at least one pharmaceutically acceptable solubility-enhancing polymer; and a CR coating comprising a water insoluble polymer alone or a water insoluble polymer in combination with a water-soluble polymer; wherein the active pharmaceutical ingredient comprises a weakly basic active pharmaceutical ingredient having a solubility of not more than 100 μg/mL at pH 6.8 (e.g., meclizine).

In yet another embodiment, the present invention is directed to a method of preparing a pharmaceutical composition comprising dissolving meclizine or a pharmaceutically acceptable salt thereof, and sufficient solubility-enhancing polymer in a pharmaceutically acceptable solvent mixture; spray coating the coating formulation onto pharmaceutically acceptable inert cores, thereby forming a solid dispersion of meclizine in the solubility-enhancing polymer on the inert core; dissolving a water insoluble polymer and an optional enteric polymer in a pharmaceutically acceptable solvent mixture and coating the solid dispersion coated core, thereby forming CR beads comprising a CR coating formed on the solid dispersion.

In yet another embodiment, the present invention is directed to a dosage form further comprising:

(a) a plurality of rapidly-dispersing microgranules each having an average particle size of not more than about 400 μm and comprising (i) a disintegrant and (ii) a sugar alcohol and/or a saccharide, wherein said sugar alcohol and/or saccharide each have an average particle size of not more than about 30 μm; and

(b) a drug core comprising meclizine 2HCl.H₂O and a polymeric binder disposed over an inert core and further comprising a taste-masking layer comprising a water insoluble polymer or a water insoluble polymer in combination with a gastrosoluble organic, inorganic or polymeric pore-former;
wherein said dosage form is an orally disintegrating tablet.

In still another embodiment, the present invention is directed to a method of treating vertigo and other diseases, comprising administering a therapeutic amount of the composition of the present invention to a patient in need thereof.

### BRIEF DESCRIPTION OF THE DRAWINGS

FIG. 1 illustrates the cross-section of a controlled-release (CR) bead in certain embodiments of the present invention: (10)—CR Bead comprising a sustained-release (SR) coating layer or a sustained-release (SR) coating followed by an optional delayed-release (DR) or timed, pulsatile release (TPR) coating layer, (9), disposed over an optional protective seal coating layer (7), disposed over meclizine drug layer (a member of the class of weakly basic, piperazine-derivatives of H₁-receptor antagonists used as an antivertigo/antiemetic agent), 5, disposed over a SR or TPR coating layer, 3, disposed over a pharmaceutically acceptable solubility-enhancing organic acid core (e.g., fumaric acid crystal or a fumaric acid layer disposed over an inert core), 1.

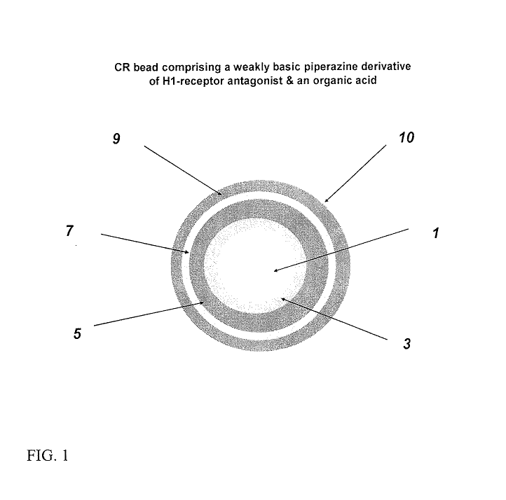

FIG. 2 illustrates the cross-section of a controlled-release (CR) bead in certain embodiments of the present invention: (20)—CR Bead comprising an compressible coating layer (19), disposed over a CR coating layer (a SR coating layer, a SR coating followed by an optional DR coating or TPR coating), 17, an optional protective seal coating layer (15) that is disposed over a solid dispersion layer (13) comprising meclizine HCl and a pharmaceutically acceptable solubility-enhancing/crystallization-inhibiting water-soluble polymer (e.g., poly(vinyl pyrrolidone-co-vinyl acetate) available as Kollidon VA 64), disposed over a pharmaceutically acceptable inert core such as sugar sphere (11).

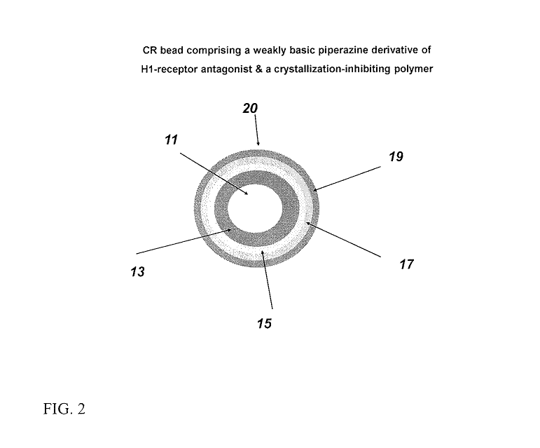

### DETAILED DESCRIPTION OF THE INVENTION

The following description includes information that may be useful in understanding the present invention. It is not an admission that any of the information provided herein is prior art or that any publication specifically or implicitly referenced is prior art.

All documents cited herein are incorporated by reference in their entirety for all purposes to the same extent as if each individual document was specifically and individually indicated to be incorporated by reference.

The term “drug”, “active”, “active agent”, or “active pharmaceutical ingredient” as used herein includes a pharmaceutically acceptable and therapeutically effective compound, pharmaceutically acceptable salts, stereoisomers and mixtures of stereoisomers, solvates (including hydrates), polymorphs, and/or esters thereof. Unless otherwise indicated, when referring to a drug in the descriptions of the various embodiments of the invention, the reference encompasses the base drug, pharmaceutically acceptable salts, stereoisomers and mixtures of stereoisomers, solvates (including hydrates), polymorphs, and/or esters thereof.

The term “salts” refers to the product formed by the reaction of a suitable inorganic or organic acid with the “free base” form of the drug. Suitable acids include those having sufficient acidity to form a stable salt, for example acids with low toxicity, such as the salts approved for use in humans or animals. Non-limiting examples of acids which may be used to form salts of meclizine include inorganic acids, e.g., HF, HCl, HBr, HI, H₂SO₄, H₃PO₄; non-limiting examples of organic acids include organic sulfonic acids, such as C₆-₁₆ aryl sulfonic acids, C₆₋₁₆ heteroaryl sulfonic acids or C₁₋₁₆ alkyl sulfonic acids—e.g., phenyl, a-naphthyl, β-naphthyl, (S)-camphor, methyl, ethyl, n-propyl, i-propyl, n-butyl, s-butyl, i-butyl, t-butyl, pentyl and hexyl sulfonic acids; non-limiting examples of organic acids includes carboxylic acids such as C₁₋₁₆ alkyl, C₆₋₁₆ aryl carboxylic acids and C₄₋₁₆ heteroaryl carboxylic acids, e.g., acetic, glycolic, lactic, pyruvic, malonic, glutaric, tartaric, citric, fumaric, succinic, malic, maleic, hydroxymaleic, benzoic, hydroxybenzoic, phenylacetic, cinnamic, salicylic and 2-phenoxybenzoic acids; non-limiting examples of organic acids include amino acids, e.g. the naturally-occurring amino acids, lysine, arginine, glutamic acid, glycine, serine, threonine, alanine, isoleucine, leucine, etc. Other suitable salts can be found in, e.g., S. M. Birge et al., J. Pharm. Sci., 1977, 66, pp. 1-19 (herein incorporated by reference for all purposes). In most embodiments, “salts” refers to salts which are biologically compatible or pharmaceutically acceptable or non-toxic, particularly for mammalian cells. The salts of drugs useful in the present invention may be crystalline or amorphous, or mixtures of different crystalline forms and/or mixtures of crystalline and amorphous forms.

As used herein, the terms “solubility-enhancing organic acid” or “solubility-modulating organic acid” refer to a water-soluble, pharmaceutically acceptable organic acid which is capable of increasing the rate and/or the extent of dissolution of the active pharmaceutical ingredient in an aqueous solution of the organic acid.

The terms “solid dispersion” or “solid solution” refer to a substantially amorphous meclizine or its salt and at least one crystallization-inhibiting polymer substantially molecularly dispersed in the solid state. The term “substantially amorphous” means that less than about 40% of the active pharmaceutical ingredient forms a separate crystalline phase in the polymeric matrix. In other embodiments, “substantially amorphous” means that less than about 30%, less than about 20%, less than about 10%, less than about 5%, or less than about 1% of meclizine forms a separate crystalline phase in the polymeric matrix. Alternatively stated, at least about 60%, at least about 70%, at least about 80%, at least about 90%, at least about 95%, or at least about 99% of the active pharmaceutical ingredient (meclizine or its salt) is in the amorphous state. The term “substantially molecularly dispersed” means that less than about 40% of the active pharmaceutical ingredient forms a separate crystalline phase in the polymeric matrix, and the remainder of the active pharmaceutical ingredient exists in the non-crystalline form in the polymeric matrix. In other embodiments, “substantially molecularly dispersed” means that less than about 30%, less than about 20%, less than about 10%, less than about 5%, or less than about 1% of the active pharmaceutical ingredient forms a separate crystalline phase in the polymeric matrix. The solid dispersions of the present invention include combinations of “substantially molecularly dispersed” and “substantially amorphous” active pharmaceutical ingredient in the polymeric matrix, provided that no more than about 40% of the active pharmaceutical ingredient, and in some embodiments or more than about 30%, no more than about 20%, or more than about 10%, no more than about 5%, or no more than about 1% of the active pharmaceutical ingredient forms a crystalline phase in the polymeric matrix.

As used herein, the terms “solubility-enhancing polymer” or “crystallization-inhibiting polymer” refers to a water-soluble polymer capable, at suitable concentrations, of forming a solid dispersion, as defined herein, of a weakly basic meclizine in the solubility-enhancing polymer, for example by first dissolving both the drug and polymer in the same solvent system, and then removing the solvent under appropriate conditions. The weakly basic drug is maintained substantially as a molecular dispersion or in amorphous form during storage, transportation, and commercial distribution of the composition containing the solid dispersion of the solubility-enhancing polymer and weakly basic drug.

The term “about” is used herein to refer to a numerical quantity, and includes “exactly”. For example, “about 60 seconds” includes 60 seconds, exactly, as well as values close to 60 seconds (e.g., 50 seconds, 55 seconds, 59 seconds, 61 seconds, 65 seconds, 70 seconds, etc.).

As used herein, the term “controlled-release” coating encompasses coatings that delay release, sustain release, prevent release, and/or otherwise prolong the release of a drug from a particle coated with a controlled-release coating. The term “controlled-release” encompasses “sustained-release,” “delayed release” and “timed, pulsatile release”, thus a “controlled-release coating” encompasses a sustained release coating, timed, pulsatile release coating or “lag-time” coating.

The term “pH sensitive” as used herein refers to polymers which exhibit pH dependent solubility.

The term “enteric polymer”, as used herein, refers to a pH sensitive polymer that is resistant to gastric juice (i.e., relatively insoluble at the low pH levels found in the stomach), and which dissolves at the higher pH levels found in the intestinal tract.

As used herein, the term “immediate release” (in reference to a pharmaceutical composition which can be a dosage form or a component of a dosage form), refers to a pharmaceutical composition which releases greater than or equal to about 50% of the active, in another embodiment greater than about 75% of the active, in another embodiment greater than about 90% of the active, and in other embodiments greater than about 95% of the active within about 2 hours, or within about one hour following administration of the dosage form. The term can also refer to pharmaceutical compositions in which the relatively rapid release of active occurs after a “lag time” (in which little or no release of active occurs).

The term “immediate release (IR) bead” or “immediate release particle” refers broadly to a bead or particle containing a member of the piperazine class of H₁-receptor antagonists, —which exhibits “immediate release” properties with respect to the drug as described herein.

The term “sustained release (SR) bead” or “sustained release particle” refers broadly to a bead or particle comprising an SR coating, as described herein, disposed over a drug-containing core coated with an SR coating as described herein.

The term “lag-time coating” or “TPR (timed, pulsatile release) coating” refers to a controlled-release coating comprising the combination of water-insoluble and enteric polymers as used herein. A TPR coating by itself provides an immediate release pulse of the drug after a predetermined lag-time. A TSR (timed, sustained release) bead with a TPR coating disposed over a barrier coating (SR coating) provides a sustained drug-release profile after a predetermined lag time.

The term “delayed release (DR) bead” or “delayed release particle” refers broadly to a drug-containing core (e.g., containing a weakly basic, piperazine derivative of H₁-receptor antagonists) coated with a DR coating as described herein. A DR coating refers to a controlled-release coating comprising an enteric polymer, optionally in combination with a plasticizer.

The term “controlled release (CR) bead” or “controlled release particle” refers broadly to a drug-containing core (e.g., containing a weakly basic, piperazine derivative of H₁-receptor antagonists) having an inner SR coating optionally followed by an outer DR or TPR coating or an inner TPR coating followed by an outer DR coating, as described herein.

The term “lag-time” as used herein refers to a time period wherein less than about 10% of the active is released from a pharmaceutical composition after ingestion of the pharmaceutical composition (or a dosage form comprising the pharmaceutical composition), or after exposure of the pharmaceutical composition, or dosage form comprising the pharmaceutical composition, to simulated body fluid(s), for example evaluated with a USP apparatus using a two-stage dissolution medium (first 2 hours in 700 mL of 0.1N HCl at 37° C. followed by dissolution testing at pH=6.8 obtained by the addition of 200 mL of a pH modifier).

The term “disposed over”, e.g. in reference to a coating over a substrate, refers to the relative location of e.g. the coating in reference to the substrate, but does not require that the coating be in direct contact with the substrate. For example, a first coating “disposed over” a substrate can be in direct contact with the substrate, or one or more intervening materials or coatings can be interposed between the first coating and the substrate. In other words, for example, a SR coating disposed over a drug-containing core can refer to a SR coating deposited directly over the drug-containing core or acid crystal or acid-containing core, or can refer to a SR coating deposited onto a protective seal coating deposited on the drug-containing core.

The term “sealant layer” or “protective seal coating” refers to a protective membrane disposed over a drug-containing core particle or a functional polymer coating. The sealant layer protects the particle from abrasion and attrition during handling, and/or minimizes static during processing.

The terms “orally disintegrating tablet” or “ODT” refers to a tablet which disintegrates rapidly in the oral cavity of a patient after administration, without the need for chewing. The rate of disintegration can vary, but is faster than the rate of disintegration of conventional solid dosage forms (e.g., tablets or capsules) which are intended to be swallowed immediately after administration, or faster than the rate of disintegration of chewable solid dosage forms, when tested as described herein (e.g. the USP <701> test method).

The term “substantially disintegrates” refers to a level of disintegration amounting to disintegration of at least about 50%, at least about 60%, at least about 70%, at least about 80%, at least about 90%, or about 100% disintegration. The term “disintegration” is distinguished from the term “dissolution”, in that “disintegration” refers to the breaking up of or loss of structural cohesion of e.g. the constituent particles comprising a tablet, whereas “dissolution” refers to the solubilization of a solid in a liquid (e.g., the solubilization of a drug in solvents or gastric fluids).

The compositions of the present invention comprise a plurality of particles containing a member of the class of weakly basic, piperazine-derivative H₁-receptor antagonists (e.g., meclizine and pharmaceutically acceptable salts thereof), comprising drug-containing core coated with a first and second coating as described herein, wherein the first coating comprises at least one water-insoluble polymer. The first coating can be disposed directly on the drug-containing core, coated onto a sealant layer which is disposed over the drug-containing core, coated over the second coating, coated over a sealant layer which is disposed over the second coating, etc.

The term “water-insoluble polymer” refers to a polymer which is insoluble or very sparingly soluble in aqueous media, independent of pH, or over a broad pH range (e.g., pH 0 to pH 14). A polymer that swells but does not dissolve in aqueous media can be “water-insoluble,” as used herein.

The term “water-soluble polymer” refers to a polymer which is soluble (i.e., a significant amount dissolves) in aqueous media, independent of pH.

The term “enteric polymer” refers to a polymer which is soluble (i.e., a significant amount dissolves) under intestinal conditions; i.e., in aqueous media under˜neutral to alkaline conditions and insoluble under acidic conditions (i.e., low pH).

The term “reverse enteric polymer” or “gastrosoluble polymer” refers to a polymer that is soluble under acidic conditions and insoluble under neutral (as in water) and alkaline conditions.

The terms “plasma concentration-time profile”, “Cₘₐₓ”, “AUC”, “T_{max”}, elimination half life” have their generally accepted meanings as defined in the FDA Guidance for Industry: Bioavailability and Bioequivalence Studies for Orally Administered Drug Products—General Considerations (issued March 2003).

Unless stated otherwise, the amount of the various coatings or layers described herein (the “coating weight”) is expressed as the percentage weight gain of the particles or beads provided by the dried coating, relative to the initial weight of the particles or beads prior to coating. Thus, a 10% coating weight refers to a dried coating which increases the weight of a particle by 10%.

As used herein, the terms “a member of the piperazine class of antihistamines”, and “piperazine derivatives of antihistamines” refer to a weakly basic, piperazine derivatives of H₁-receptor antagonists.

Non-adherence to dosing regimens is a major medical problem in the world costing billions of dollars and affecting lifestyles of millions of people. In addition to a properly designed drug delivery system, the time of administration is equally important. However, there are several challenges to be overcome before an acceptable solution can be found. Factors known to limit drug absorption of orally administered weakly basic piperazine derivatives of H₁-receptor antagonists Which can have widely varying biopharmaceutical, physicochemical, and organoleptic properties, including varying minimum therapeutically effective doses), include poor pH dependent solubility, inadequate stability in GI fluids, poor permeability across the intestinal epithelium, enzymatic degradation/metabolism in certain segments, and complexation or high protein binding. While the orally administered pharmaceutical dosage form passes through the human digestive tract, the drug should be released from the dosage form and be available in solution form at or near the absorption site in the gastrointestinal (GI) tract for absorption to occur. The rate at which the drug goes into solution and is released from a dosage form is important from the kinetics of drug absorption. The dosage form and hence the active ingredient is subjected to varying pH levels during the transit, i.e., pH values varying from about 1.2 (stomach pH during fasting but may vary between 1.2 and 4.0 upon consumption of food) to about 7.4 (bile pH: 7.0-7.4 and intestinal pH: 5 to 7). Moreover, the transit time of a dosage form in individual parts of the digestive tract, especially the gastric residence time, may vary significantly depending on its size and prevailing local conditions. Furthermore, the fluid volume in individual parts of the digestive tract varies significantly [e.g., stomach: (fasted: 46 mL) and (fed: 686 mL); small intestine: (fasted: 105 mL) and (fed: 54 mL)) and colon: (fasted: 13 mL) and (fed: 11 mL)]. The surface area available for drug absorption also varies significantly in different parts of the GI tract (400-650 cm long small intestine with plicae circulares and villi accounting for large surface area vs. 120 cm long large intestine). After oral administration, different drugs are affected by the biochemical processes of absorption, distribution, metabolism, and elimination (ADME) differently. For example, different drugs of the same therapeutic class may be absorbed into the bloodstream at different rates and sometimes through different processes. The rate and extent of absorption for a particular drug, and among different drugs, may vary along the GI tract. For example many drugs are absorbed faster and to a greater extent in the small intestine than in the large intestine. The drug absorbed into the bloodstream may be rapidly distributed in the peripheral tissues, metabolized (e.g., oxidizing, hydrolyzed, and/or conjugated by enzymes n the liver, epithelial cells in gut wall producing sometimes active metabolites, and eventually eliminated/excreted from the body via kidney in urine or in bile acids via liver into the GI tract for hepatic recirculation or excretion in feces. Another drug characteristic that affects the feasibility of developing extended release dosage form is its elimination half-life, which refers to the time required to reduce the plasma concentration of the drug by 50% of its time zero value.

Pharmacokinetic modeling is typically constructed by fitting the plasma concentration-time data from intravenous and immediate release (IR) peroral dosage forms of a weakly basic pharmaceutical active of interest, and/or pharmacokinetic constants, such as absorption constant (Kₐ), bioavailability (F), volume of distribution (V), rate constants (K₁₂, K_{2,1}) to and from the peripheral compartment, distribution rate constant (a per hr), elimination rate constant (β per hr), and lag time (T_{lag}), using a PK/PD simulation software, WinNonlin® from Pharsight® Corporation (Mountain View, Calif.) and/or GastroPlus™ from Simulationsplus, Inc., so that the predicted plasma concentration-time profiles closely match the actual plasma concentration-time profiles reported in the literature. Thereafter fitting and parameter adjustments are performed to identify target in vivo plasma concentration-time profiles suitable for a once-daily dosing regimen and desired in vitro sustained release profiles are deconvoluted assuming one or two compartment models and first order absorption/elimination, linear pharmacokinetics, and in vitro/in vivo correlations.

A set of theoretical release rates (K₁ values) would then be used in the in the simulations to determine potential (desired or target) in vitro drug release profiles such that simulated AUC (0-24 hr) values for a variety of formulations would be estimated for comparison with that of immediate release reference listed drug product, prototypes for the weakly basic, piperazine derivative of H₁-receptor antagonists of interest would be designed based on strategic approaches (e.g., Diffucaps® approach, organic acid approach, and/or solid solution approach) for evaluation in a comparative human PK study to confirm in vitro/in vivo correlations. Further clinical studies are required to confirm and/or establish PK/PD relationships for the same weakly basic drug.

It was surprisingly discovered that the probability of successfully developing once-daily drug delivery systems containing a weakly basic piperazine derivative can be increased either by incorporating a solubility-enhancing organic acid such as fumaric acid or a crystallization-inhibiting water-soluble polymer such as pyrrolidone-vinyl acetate copolymer (commercially available from BASF as Kollidon VA 64). Without going into the mechanism of drug solubilization and release, it is postulated that when an organic acid is used, the acid creates an acidic pH microenvironment within the CR coated drug particle in which the drug is soluble prior to releasing it to a hostile alkaline pH environment where the weakly basic drug is practically insoluble. When a crystallization-inhibiting polymer is dissolved along with the weakly basic drug in a common solvent mixture, the solid solution/dispersion so produced creates and maintains the drug in the amorphous form which is significantly more soluble irrespective of the physiological pH allows prolonged drug release from CR coated beads.

In certain embodiments, the present invention is directed to a controlled release composition comprising a plurality of particles comprising a pharmaceutically acceptable solubility-enhancing organic acid or water-soluble polymer and a weakly basic, piperazine derivative of H₁-receptor antagonists, such as meclizine or its salt, which can be used as an antivertigo/antiemetic agent in the management of nausea, vomiting, and dizziness associated with motion sickness and vertigo in diseases affecting the vestibular apparatus. Each of the drug-containing particles comprises a core comprising a weakly basic drug such as meclizine or meclizine 2HCl.H₂O and a solubility enhancing organic acid or a crystallization-inhibiting water-soluble polymer, and is coated with one or more functional polymer coatings which impart the desired extended release properties. The drug-containing core comprises a pharmaceutically acceptable organic acid crystal or an organic acid layer disposed over an inert core and coated with one or more functional polymer coatings which impart the desired extended release properties. The first coating disposed over the organic acid core comprises at least one water-insoluble polymer, and the second optional coating disposed over the first SR coating layer comprises an enteric polymer and an optional water-insoluble polymer. The first and second coatings can be applied in any order. Further, the first coating comprising a water insoluble polymer is disposed over the weakly basic drug layer, followed by the second coating comprising an enteric polymer optionally in combination with a water insoluble polymer. Alternatively, the first coating comprises a combination of enteric and water insoluble polymers applied over the core particle containing a weakly basic, piperazine derivative of an H₁-receptor antagonist, which is followed by a second delayed release coating. Other coatings in addition to the first and second coating can also be applied (e.g., seal coatings or other extended release coatings) in any order, i.e., prior to, between, or after either of the first and second coatings.

Suitable weakly basic drugs of the piperazine class of H₁-receptor antagonists include, for example, buclizine, cinnarizine, cyclizine, hydroxyzine, meclizine, niaprazine and salts thereof, and the like.

In one embodiment, the pharmaceutical compositions of the present invention comprise a plurality of CR and IR particles, wherein the CR particles each comprises a core coated with a water-insoluble polymer layer, followed by a coating layer comprising an enteric polymer optionally in combination with a water-insoluble polymer; wherein the core comprises a weakly basic, piperazine derivative of H₁-receptor antagonists (e.g. meclizine) and a pharmaceutically acceptable polymeric binder, followed by a first coating layer comprising a water-insoluble polymer optionally in combination with a water-soluble polymer and an optional second coating of an enteric polymer optionally in combination with a water-insoluble polymer.

In another embodiment, the pharmaceutical compositions of the present invention comprise a plurality of CR and IR particles, wherein each CR particle comprises a core coated with a water-insoluble polymer layer, followed by a coating layer comprising an enteric polymer optionally in combination with a water-insoluble polymer; the core comprises a weakly basic, piperazine derivative of H₁-receptor antagonists (e.g. meclizine) and a pharmaceutically acceptable organic acid (e.g. fumaric acid) separated from each other at least by a SR layer; and the IR particles each comprise the piperazine derivative in combination with suitable excipients. In certain embodiments, the pharmaceutical composition comprises a weakly basic pharmacological agent and at least one solubility-enhancing organic acid which is capable of creating an acidic pH microenvironment within the coated bead to solubilize the weakly basic drug prior to its release into a hostile pH environment of the intestinal tract wherein the drug is practically insoluble, in accordance with the specifications co-pending U.S. patent application Ser. No. 11/668,167 (published as US 2007/0196491 A1), No. 11/668,408 (published as US 2007/0190145 A1) and No. 12/209,285 (published as US 2009/0232885 A1). Each of these applications set forth herein are incorporated by reference in their entireties for all purposes.

In a particular embodiment, the CR particles comprise an inert core (e.g., a sugar sphere, cellulosic sphere etc.) sequentially coated with a pharmaceutically acceptable organic acid (e.g., succinic acid) and a pharmaceutically acceptable binder (e.g., hydroxypropyl cellulose); a sustained release (SR) layer (e.g., comprising a pharmaceutically acceptable water insoluble polymer such as ethyl cellulose, optionally plasticized with a pharmaceutically acceptable plasticizer such as triethyl citrate or polyethylene glycol); a drug layer comprising the piperazine-derivative of H₁-receptor antagonists (e.g. meclizine or a pharmaceutically acceptable salt and/or solvate thereof) and a pharmaceutically acceptable binder (e.g., povidone); an optional sealing layer (e.g. comprising a water soluble polymer such as hydroxypropyl methyl cellulose); and a SR layer comprising a water insoluble polymer such as ethyl cellulose (EC-10), and an enteric polymer such as hypromellose phthalate, HP-55, and an optional pharmaceutically acceptable plasticizer such as triethyl citrate (TEC).

In another embodiment, the present invention is directed to a dosage form comprising:

(a) a core comprising an organic acid crystal (e.g., aspartic acid) with a desired mean particle size;

(b) a first coating disposed over the acid crystal comprising at least one water-insoluble polymer;

(c) a second coating disposed over the first coating comprising an enteric polymer optionally in combination with a water-insoluble polymer;

(d) a third coating disposed over the second coating comprising meclizine hydrochloride and a polymeric binder;

(e) a fourth coating disposed over said meclizine hydrochloride layer comprising at least one water-insoluble polymer; and

(f) a fifth coating disposed over said fourth coating comprising an enteric polymer optionally in combination with a water-insoluble polymer.

A non-limiting list of pharmaceutically acceptable organic acids includes citric acid, lactic acid, fumaric acid, malic acid, maleic acid, tartaric acid, succinic acid, oxalic acid, aspartic acid, and glutamic acid. In a particular embodiment, the pharmaceutically acceptable organic acid is fumaric acid.

Alternatively, the pharmaceutical compositions of most embodiments comprise a weakly basic drug (e.g., meclizine and pharmaceutically acceptable salts thereof) and at least one solubility-enhancing/crystallization inhibiting water-soluble polymer which is capable of creating and maintaining the weakly basic drug in the amorphous form wherein the amorphous drug is more soluble, in accordance with the specifications co-pending U.S. patent application Ser. No. 11/847,219 (published as US 2008/0069878 A1).

The present invention is also directed to pharmaceutical compositions comprising the combination of a solid dispersion of a weakly basic, piperazine derivative of an H₁-receptor antagonists, such as meclizine or a pharmaceutically acceptable salt, isomer, hydrate, and a mixture thereof and at least one solubility-enhancing/crystallization-inhibiting polymer such as Kollidon VA 64 (polyvinylpyrrolidone-co-vinylacetate), with a controlled-release (CR) coating comprising a water-insoluble polymer alone, a water-insoluble polymer in combination with an optional water-soluble or enteric polymer. The composition of the solid dispersion of meclizine with a CR coating provides an improved release profile compared to the release profile obtained by conventional compositions in which the weakly basic drug is not present in the form of a solid dispersion and/or which lacks a CR coating. For example, by suitable manipulation of the composition comprising at least one CR coating the drug release can be prolonged over 12-18 hours, or the time to reach Cₘₐₓ (i.e., maximum plasma concentration) can be delayed relative to using the solubility-enhancing polymer alone.

An inert core thus coated with a drug layer, and lacking extended release coatings has immediate release properties, and can be referred to as an “IR bead” or a “rapid release bead”. Depending on the characteristics of the specific piperazine-derivative of H₁-receptor antagonists, the drug can be deposited from solution directly onto the inert core or a coated organic acid core or crystal without using a binder. In various other embodiments, the drug layer contains a binder (typically a pharmaceutically acceptable water-soluble polymer) that facilitates the binding of the drug to the inert sugar sphere.

Examples of suitable binders include, but are not limited to, polyvinylpyrrolidone (PVP), polyethylene oxide, hydroxypropyl methyl cellulose (HPMC), hydroxypropylcellulose (HPC), and polysaccharides. The binder can be present in an amount ranging from about 0.5 to about 10 weight % based on the total weight of the drug layer.

The drug layer is typically deposited by spraying a drug and optionally binder containing solution onto the inert cores, e.g., using a fluidized bed coating apparatus. The drug layering solution comprises a pharmaceutically acceptable solvent in which the piperazine-derivative of H₁-receptor antagonists and optional binder are dissolved. In some embodiments, the piperazine-derivative of H₁-receptor antagonists may be present in the form of a suspension. Depending on the viscosity, the solids content of the drug-layering solution may be up to about 35 weight %, for example about 10%, about 15%, about 20%, about 25%, about 30%, etc. Pharmaceutically acceptable solvents include water, alcohols (such as ethanol), acetone, etc.

Alternatively, the core containing a piperazine-derivative of H₁-receptor antagonists can be a granulate comprising the drug in combination with one or more pharmaceutically acceptable excipients (e.g., lactose, mannitol, microcrystalline cellulose, etc.). Such granulates can be prepared by conventional granulation methods, and may optionally include suitable binders as described herein.

In one embodiment, the core containing the solid dispersion is prepared by granulating the solubility enhancing polymer, the weakly basic drug and optionally other pharmaceutically acceptable excipients (e.g., binders, diluents, fillers) in a high-shear granulator, or a fluid bed granulator, such as Glatt GPCG granulator, and granulated to form agglomerates. The wet mass from the high-shear granulator can also be extruded and spheronized to produce spherical particles (pellets).

When pharmaceutical compositions of the present invention are formulated into an ODT dosage form, the compositions further comprise rapidly dispersing microgranules. The rapidly dispersing microgranules comprise at least one disintegrant in combination with at least one sugar alcohol and/or saccharide. Non-limiting examples of suitable disintegrants include crospovidone (crosslinked polyvinylpyrrolidone), starch, low-substituted hydroxypropylcellulose, sodium starch glycolate, and crosslinked sodium carboxymethyl cellulose. Non-limiting examples of sugar alcohols include arabitol, erythritol, lactitol, maltitol, mannitol, sorbitol, and xylitol. Non-limiting examples of suitable saccharides include lactose, sucrose, and maltose. The ratio of the disintegrant to the sugar alcohol and/or saccharide in the rapidly dispersing microgranules ranges from about 1/99 to about 10/90, and in some embodiments is about 5/95 (by weight).

Since ODT dosage forms disintegrate rapidly in the oral cavity of a patient, the organoleptic properties of the ODT are an important consideration. For example, the ODT should be formulated to provide good “mouthfeel” and taste characteristics. “Mouthfeel” describes how a product feels in the mouth. In order to obtain a “mouthfeel” which is not gritty, the CR beads, rapidly dispersing microgranules, and optional IR beads should have an average particle size of about 400 μm or less, in some embodiments about 300 μm or less, and in still other embodiments, about 200 μm or less. In one embodiment, the primary particles comprising the rapidly dispersing microgranules (i.e., particles of a disintegrant and sugar alcohol and/or saccharide which are agglomerated to form the rapidly dispersing microgranules) have an average particle size of about 30 μm or less, in other embodiments about 25 μm or less, and in still other embodiments about 20 μm or less. Rapidly dispersing granules comprising a sugar alcohol and/or saccharide having an average particle size of less than about 30 μm provide superior oral disintegration properties compared to granules comprising larger average particle sizes of sugar alcohol or saccharide. The combination of less than about 30 μm sugar alcohol and/or saccharide particles with particular disintegrants (e.g., crospovidone, crosslinked sodium carboxymethyl cellulose, and low-substituted hydroxypropylcellulose) provides particularly good disintegration properties.

When the dosage forms of the present invention are formulated as orally disintegrating tablets comprising both IR and CR beads, the IR beads can be uncoated, optionally coated with a sealant or protective coating, and/or optionally coated with a taste masking layer depending on the organoleptic properties of the piperazine derivative H₁-receptor antagonist. The taste masking layer can include e.g. any of the taste masking compositions described in U.S. Application Publication Nos. US 2006/0105038, US 2006/0078614, and US 2006/0105039, each of which is herein incorporated by reference in its entirety. Specifically, suitable taste masking layers comprise one or more pharmaceutically acceptable water-insoluble polymers combined with one or more pore forming agents. Non-limiting examples of suitable pharmaceutically acceptable water-insoluble polymers for the taste masking layer include, e.g. ethyl cellulose, cellulose acetate, cellulose acetate butyrate, polyvinyl acetate, and methacrylate polymers (e.g., Eudragit RL, RS, NE 30D). Eudragit RL and RS are copolymers of ethyl acrylate, methyl methacrylate, and has a low content of methacrylic acid ester with quaternary ammonium groups. Eudragit NE is a neutral copolymer comprising ethyl acrylate and methyl methacrylate. Non-limiting examples of suitable pore forming agents include sodium chloride, calcium carbonate, calcium phosphate, calcium saccharide, calcium succinate, calcium tartrate, ferric acetate, ferric hydroxide, ferric phosphate, magnesium carbonate, magnesium citrate, magnesium hydroxide, magnesium phosphate, polyvinyl pyrrolidone, crospovidone, Eudragit E100, Eudragit EPO, and mixtures thereof. The ratio of water-insoluble polymer to pore former in the taste masking layer ranges from about 95/5 to about 50/50, or in some embodiments about 85/15 to about 65/35. The amount of taste masking layer applied to the IR bead can range from about 5% to about 50% of the total weight of the coated IR bead, in some embodiments about 10% to about 50% of the total weight of the coated IR bead.

The ratio of coated drug-containing beads (i.e., coated beads comprising the solid solution) to rapidly dispersing microgranules in the ODT dosage form varies from about 1/9 to 1/1 and in some embodiments from about 1:4 to about 1:2.

The core containing a piperazine-derivative of H₁-receptor antagonists of the present invention has an average particle size of not more than about 2 mm in some embodiments if to be filled into a hard gelatin capsule, or not more than about 400 μm in other embodiments, not more than about 300 μm in some other embodiments and not more than about 200 μm in yet other embodiments, if intended to be incorporated into an ODT.

In one embodiment, the first coating comprising the water-insoluble polymer is coated onto the core containing a piperazine-derivative of H₁-receptor antagonists (wherein the core is optionally coated with a sealant layer), thereby providing a sustained release (SR) coating.

Non-limiting examples of suitable water-insoluble polymers include ethyl cellulose, cellulose acetate, cellulose acetate butyrate, polyvinyl acetate, neutral copolymers of acrylate/methacrylate esters (e.g., Eudragit NE, which is a copolymer of ethyl acrylate and methyl methacrylate), waxes, and mixtures thereof. In a particular embodiment, the water-insoluble polymer comprises ethyl cellulose. In another particular embodiment, the water-insoluble polymer comprises ethyl cellulose with a mean viscosity of 10 cps in a 5% solution in 80/20 toluene/alcohol measured at 25° C. on an Ubbelohde viscometer.

Suitable coating weights for the first SR coating disposed over the organic acid core or the weakly basic, piperazine drug layer comprising a water-insoluble polymer range from about 3% to about 40%, including about 3%, about 5%, about 7%, about 10%, about 12%, about 15%, about 17%, about 20%, about 22%, about 25%, about 27%, about 30%, about 35%, and about 40%, inclusive of all ranges and subranges therebetween.

In some embodiments, the water-insoluble polymer provides suitable properties (e.g., extended release characteristics, mechanical properties, and coating properties) without the need for a plasticizer. For example, coatings comprising polyvinyl acetate (PVA), neutral copolymers of acrylate/methacrylate esters such as commercially available Eudragit NE30D from Evonik Industries, ethyl cellulose in combination with hydroxypropylcellulose, waxes, etc. can be applied without plasticizers.

In yet another embodiment, the water-insoluble polymer may include a plasticizer. The amount of plasticizer required depends upon the plasticizer, the properties of the water-insoluble polymer, and the ultimate desired properties of the coating. Suitable levels of plasticizer range from about 1% to about 20%, from about 3% to about 20%, about 3% to about 5%, about 7% to about 10%, about 12% to about 15%, about 17% to about 20%, or about 1%, about 2%, about 3%, about 4%, about 5%, about 6%, about 7%, about 8%, about 9%, about 10%, about 15%, or about 20% by weight relative to the total weight of the coating, inclusive of all ranges and subranges therebetween.

Non-limiting examples of suitable plasticizers include triacetin, citrate esters, triethyl citrate, acetyltriethyl citrate, tributyl citrate, acetyl tri-n-butyl citrate, diethyl phthalate, dibutyl phthalate, dioctyl phthalate, methyl paraben, propyl paraben, propyl paraben, butyl paraben, dibutyl sebacate, substituted triglycerides and glycerides, monoacetylated and diacetylated glycerides (e.g., Myvacet® 9-45), glyceryl monostearate, glycerol tributyrate, polysorbate 80, polyethylene glycol, propylene glycol, oils (e.g. castor oil, hydrogenated castor oil, rape seed oil, sesame oil, olive oil, etc.), glycerin sorbitol, diethyl oxalate, diethyl malate, diethyl fumarate, diethylmalonate, dibutyl succinate, fatty acids, and mixtures thereof.

Further non-limiting examples of suitable plasticizers include glycerol and esters thereof (e.g., monoacetylated glycerides, acetylated mono- or diglycerides (e.g., Myvacet® 9-45)), glyceryl monostearate, glyceryl triacetate, glyceryl tributyrate, phthalates (e.g., dibutyl phthalate, diethyl phthalate, dimethyl phthalate, dioctyl phthalate), citrates (e.g., acetylcitric acid tributyl ester, acetylcitric acid triethyl ester, tributyl citrate, acetyltributyl citrate, triethyl citrate), glyceroltributyrate; sebacates (e.g., diethyl sebacate, dibutyl sebacate), adipates, azelates, benzoates, chlorobutanol, polyethylene glycols, vegetable oils, fumarates, (e.g., diethyl fumarate), malates, (e.g., diethyl malate), oxalates (e.g., diethyl oxalate), succinates (e.g., dibutyl succinate), butyrates, cetyl alcohol esters, malonates (e.g., diethyl malonate), castor oil, and mixtures thereof. When used in an embodiment of the present invention, the plasticizer may constitute from about 3% to about 30% by weight of the polymer(s) in the controlled-release coating. In still other embodiments, the amount of plasticizer relative to the weight of the polymer(s) in the controlled-release coating is about 3%, about 5%, about 7%, about 10%, about 12%, about 15%, about 17%, about 20%, about 22%, about 25%, about 27%, and about 30%, inclusive of all ranges and subranges therebetween. One of ordinary skill in the art will recognize that the presence of plasticizer, or type(s) and amount(s) of plasticizer(s) can be selected based on the polymer or polymers and nature of the coating system (e.g., aqueous or solvent-based, solution or dispersion-based and the total solids).

In certain other embodiments, the first coating layer disposed over the organic acid core comprises an enteric polymer in combination with an optional water-insoluble polymer, thereby providing a timed pulsatile release (TPR) coating.

In various embodiments, the second coating or outer layer disposed over the SR coated piperazine-derivative of H₁-receptor antagonists core comprises an enteric polymer in combination with an optional water-insoluble polymer, thereby providing a timed pulsatile release (TPR) coating. In still other embodiments, the second coating comprises an enteric polymer disposed on the particle containing a piperazine-derivative of H₁-receptor antagonists, thereby providing a delayed release (DR) coating.

Non-limiting examples of suitable enteric polymers include cellulose acetate phthalate, hydroxypropyl methyl cellulose phthalate, hydroxypropyl methyl cellulose acetate succinate, polyvinyl acetate phthalate, pH-sensitive methacrylic acid/methylmethacrylate copolymers (e.g., Eudragit® L, S and FS polymers), shellac, and mixtures thereof. In certain embodiments, non-polymeric enteric materials such as non-polymeric waxes and fatty acid compositions may be used instead of enteric polymers, provided they have the pH sensitive solubility associate with enteric polymers. These enteric polymers may be used as a solution in a solvent mixture or an aqueous dispersion. Some commercially available materials that may be used are methacrylic acid copolymers sold under the trademark Eudragit (L100, S100, L30D) manufactured by Rohm Pharma, Cellacefate (cellulose acetate phthalate) from Eastman Chemical Co., Aquateric (cellulose acetate phthalate aqueous dispersion) from FMC Corp., and Aqoat (hydroxypropyl methyl cellulose acetate succinate aqueous dispersion) from Shin Etsu K.K.

In another embodiment, the plasticizer(s) used in the coatings on the organic acid-containing and/or drug-containing particles are free of phthalates. In another embodiment, the first coating comprising a water-insoluble polymer and optionally a water-soluble polymer comprises a plasticizer that is free of phthalates. In another embodiment, the second coating comprising an enteric polymer and optionally a water-insoluble polymer comprises a plasticizer that is free of phthalates. In another embodiment, the first and second coatings each comprise a plasticizer that is free of phthalates. In another embodiment, all of the coatings disposed over the drug core are free of phthalates.

When the first coating disposed over the organic acid core or second or outer coating disposed over the organic acid and/or piperazine-derivative of H₁-receptor antagonists comprises a water-insoluble polymer in combination with the enteric polymer (e.g., a TPR coating), the ratio of the water-insoluble polymer to enteric polymer ranges from about 10:1 to about 1:2, including the ranges of from about 9:1 to about 3:1, and from about 3:1 to about 1:1. In particular embodiments, the ratio of water-insoluble polymer to enteric polymer is about 1:1, about 1.5:1, about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1, or about 10:1, inclusive of all values, ranges, and subranges therebetween.

In most embodiments of the compositions of the present invention having a TPR coating, the TPR coating is applied at a coating weight of about 5% to about 60% by weight, including the ranges of from about 10% to about 50%, from about 20% to about 40%, and from about 25% to about 35%, or at a coating weight of about 5%, about 6%, about 7%, about 8%, about 9%, about 10%, about 12%, about 14%, about 16%, about 18%, about 20%, about 25%, about 30%, about 35%, about 40%, about 45%, or about 50%, inclusive of all ranges and subranges therebetween.

In a particular embodiment, the TPR coating comprises ethyl cellulose (e.g., EC-10) as the water-insoluble polymer and hypromellose phthalate (e.g., HP-55) as the enteric polymer.

Similar to the SR coating, DR and TPR coatings can also include one or more optional plasticizers (e.g. any of the plasticizers described herein). The amount of plasticizer required depends upon the plasticizer, the properties of the water-insoluble and/or enteric polymer(s), and the ultimate desired properties of the coating. Suitable levels of plasticizer range from about 1% to about 20%, from about 3% to about 20%, about 3% to about 5%, about 7% to about 10%, about 12% to about 15%, about 17% to about 20%, or about 1%, about 2%, about 3%, about 4%, about 5%, about 6%, about 7%, about 8%, about 9%, about 10%, about 15%, or about 20% by weight relative to the total weight of the coating, inclusive of all ranges and subranges therebetween.

The extended release compositions of the present invention may further comprise a sealant layer disposed on the particle containing a piperazine-derivative of H₁-receptor antagonists, e.g. between the first and second coatings, beneath the first and second coatings, and/or over both of the first and second coatings to prevent (or minimize) static and/or particle attrition during processing and handling.

In one embodiment, the sealant layer comprises a hydrophilic polymer. Non-limiting examples of suitable hydrophilic polymers include hydrophilic hydroxypropylcellulose (e.g., Klucel® LF), hydroxypropyl methyl cellulose or hypromellose (e.g., Opadry® Clear or Pharmacoat™ 603), vinylpyrrolidone-vinylacetate copolymer (e.g., Kollidon® VA 64 from BASF), and ethyl cellulose, e.g. low-viscosity ethyl cellulose. The sealant layer can be applied at a coating weight of about 1% to about 10%, for example about 1%, about 2%, about 3%, about 4%, about 5%, about 6%, about 7%, about 8%, about 9%, or about 10%, inclusive of all ranges and subranges therebetween.

In another embodiment, the compositions of the present invention further comprise a compressible coating disposed over the controlled-release coating (i.e., disposed on the outer-most functional coating). The compressible coating comprises a polymer, including but not limited to hydroxypropylcellulose, poly(vinyl acetate-vinyl pyrrolidone), polyvinyl acetate, ethyl cellulose (e.g., plasticized low-viscosity ethyl cellulose latex dispersions), etc. The compressible coating can be plasticized or unplasticized, and promotes the integrity of the controlled-release coating during compression. In one embodiment, the compressible coating comprises a plasticizer that is free of phthalates.

In another embodiment controlled release compositions of the present invention can further comprise rapidly disintegrating granules comprising a saccharide and/or a sugar alcohol in combination with a disintegrant. Suitable disintegrants include, but are not limited to for example, disintegrants selected from the group consisting of crospovidone, sodium starch glycolate, starch, crosslinked sodium carboxymethyl cellulose, low-substituted hydroxypropylcellulose, gums (e.g., gellan gum) and combinations thereof. Suitable saccharides and/or sugar alcohols may be selected from the group consisting of arabitol, erythritol, glycerol, hydrogenated starch hydrolysate, isomalt, lactitol, lactose, maltitol, mannitol, sorbitol, xylitol, sucrose, maltose, and combinations thereof. The saccharide and/or sugar alcohol may also be supplemented or replaced with artificial sweeteners such as sucralose. The ratio of the disintegrant to the saccharide and/or sugar alcohol in the rapidly dispersing microgranules ranges from about 1:99 to about 10:90, from about 5:95 to about 10:90 on a weight basis and inclusive of all ranges and subranges therebetween. In most embodiments, the disintegrant or the saccharide and/or sugar alcohol, or both, are present in the form of particles having an average particle size of about 30 μm or less in accordance with the specifications co-pending U.S. patent application Ser. No. 10/356,641 (published as US 2005/0215500 A1), No. 10/827,106 (published as US 2005/0232988 A1) and No. 12/166,757 (published as US 2009/0092672 A1). Each of these applications set forth herein are incorporated by reference in their entireties for all purposes. The ratio of the weakly basic, piperazine derivative-containing beads to the rapidly disintegrating granules can range from about 1:6 to about 1:2, from about 1:5 to about 1:3, or about 1:6, about 1:5, about 1:4, about 1:3, or about 1:2, inclusive of all ranges and subranges therebetween.

The multiple controlled-release coatings of the compositions of the present invention contribute to the control of dissolution at the drug interface and hence control the release of the piperazine-derivative of H₁-receptor antagonists (e.g., meclizine or salts, and/or solvates thereof) from the particles of the controlled release compositions of the present invention.

The achievable lag time, delayed release time, or sustained-release properties depend on the composition and thickness of the controlled-release coatings. Specific factors that can affect achieving optimal once-daily dosage forms include, but are not limited to, the pKa of the piperazine-derivative of H1-receptor antagonists and its solubility, e.g. in GI fluids.

The in vitro drug release data obtained particles coated with the multiple controlled release coatings described herein provide release profiles for a piperazine-derivative of H₁-receptor antagonists, which thereby provide pharmacokinetic profiles suitable for a once-daily dosing regimens. In one embodiment, the sustained-release coating provides release of an drug which is sustained over about 12 to about 16 hours when tested in the two-stage dissolution method (700 mL of 0.1N HCl (hydrochloric acid) for the first 2 hours and thereafter in 900 mL at pH 6.8 obtained by adding 200 mL of a pH modifier), suitable for a once-daily dosing regimen.

The controlled release compositions of the present invention can be formulated with optional pharmaceutically acceptable excipients (binders, a disintegrants, fillers, diluents, compression aids (e.g., microcrystalline cellulose/fused silicon dioxide), lubricants, etc.) into any suitable oral dosage form, for example sachets, tablets, capsules, or orally disintegrating tablets (ODTs). In one embodiment, the dosage form is a tablet, for example a tablet with a friability of less than about 1%. In another embodiment, the dosage form is a capsule filled with at least one population of particles comprising the controlled release composition of the present invention. The capsule can be for example, a hard gelatin or HPMC (hydroxypropylmethylcellulose) capsule.

In other embodiments, the dosage form is an ODT. ODTs of the present invention disintegrate in the oral cavity and are easily swallowed without water. For example, an ODT of the present invention substantially disintegrates within about 60 seconds after contact with saliva in the oral cavity or with simulated saliva fluid. In another embodiment, the ODT substantially disintegrates within about 30 seconds. Disintegration is tested according to the USP <701> Disintegration Test (herein incorporated by reference in its entirety for all purposes). In most embodiments, the ODT substantially disintegrates in the oral cavity of a patient, forming a smooth, easy-to-swallow suspension having no gritty mouthfeel or aftertaste, and provides a target PK profile (e.g., plasma concentration vs. time plot) of the piperazine-derivative of H₁-receptor antagonists (e.g., meclizine) suitable for a once-daily dosing regimen. For example, the ODT provides prolonged release of the piperazine-derivative of H₁-receptor antagonists over a period of 8-18 hrs. ODT formulations of the present invention are especially useful for treating geriatric patients (who often have difficulty swallowing conventional tablets and capsules) or for treating mentally ill patients (who often resist or “cheek” their medications). The administration of ODTs to geriatric and/or mentally ill patients will reduce the frequency of dosing and ease patient non-compliance issues.

In a particular embodiment, the ODT of the present invention comprises a therapeutically effective amount of meclizine or salts and/or solvates thereof. After administration, the ODT substantially disintegrates in the oral cavity of a patient, forming a smooth, easy-to-swallow suspension having no gritty mouthfeel or aftertaste, and provides a target PK profile (i.e., plasma concentration vs. time plot) of meclizine suitable for a once-daily dosing regimen. In addition to the controlled release composition of the present invention and rapidly disintegrating granules, the ODT of the present invention optionally includes pharmaceutically acceptable excipients such as compressible diluents, fillers, coloring agents, and optionally a lubricant.

In some embodiments, the ODT is not more than about 2000 mg; for example, about 2000 mg or less; about 1500 mg or less; about 1000 mg or less; about 500 mg or less. In another embodiment, the ODT weighs not more than about 1600 mg. In another embodiment, the ODT not more than about 800 mg. In another embodiment, the ODT weighs not more than about 500 mg. The dosage forms of the present invention can comprise two or more populations of particles containing a piperazine-derivative of H₁-receptor antagonists including at least one population of controlled release particles as described herein. For example, the dosage form can comprise a population of controlled release particles as described herein, and in addition, immediate release (IR) particles, for example uncoated cores comprising a weakly basic, piperazine derivative. In one embodiment, the dosage form comprising two or more populations of a weakly basic, piperazine derivative-containing particles is an ODT. When the dosage form is ODT, the two or more populations of a weakly basic, piperazine derivative-containing particles are combined with rapidly disintegrating microgranules, and the particles containing a piperazine-derivative of H₁-receptor antagonists and rapidly disintegrating microgranules have a particle size which provides a smooth, non-gritty mouth feel after disintegration of the ODT in the oral cavity. In one embodiment, an ODT of the present invention comprises either of SR, DR or CR particle populations; in another embodiment, the ODT comprises a combination of IR particles and SR particles; in yet another embodiment, the ODT comprises SR particles in combination with enteric coated TPR particles, and optionally in combination with (optionally taste-masked) IR particles (in addition to rapidly disintegrating microgranules). In yet another embodiment, an ODT of the present invention comprises enteric coated SR beads with or without a compressible coating in combination with rapidly dispersing granules (e.g., mannitol-crospovidone microgranules).

If the ODT of the present invention includes IR particles, the IR particles can be coated with a taste-masking coating which allows rapid release of the piperazine-derivative of H₁-receptor antagonists upon entry into the stomach, but prevents drug release in the oral cavity, and thus prevents any off-taste from the particles containing a piperazine-derivative of H₁-receptor antagonists. That is, a taste-masked IR particle releases not more than about 10% of the total amount of the drug contained in the IR particle in 3 minutes (the longest typical residence time anticipated for the ODT in the buccal cavity) when dissolution tested in simulated saliva fluid (pH˜6.8), while releasing not less than about 75% of the total amount drug in the IR particles in about 60 minutes when dissolution tested in 0.1N HCl.

In various embodiments of the present invention, when the dosage form comprises IR particles in addition to the controlled release particles, the ratio of IR particles to total of CR particles (e.g., a SR particle population with an enteric or TPR coating) ranges from about 0:100 (i.e., no IR particles) to about 50:50, for example from about 10:90 to about 20:80, from about 30:70 to about 40:60, or about 5:95, about 10:90, about 15:85, about 20:80, about 25:75, about 30:70, about 35:65, about 40:60, about 45:55, or about 50:50, inclusive of all ranges and subranges therebetween.

In certain embodiments of the ODT of the present invention, the ratio of the rapidly dispersing microgranules and the piperazine-derivative of H₁-receptor antagonists-containing particles (e.g. IR and/or CR particles) are at a ratio of from about 4:1 to 1:1, thereby providing a smooth mouthfeel upon administration. In a particular embodiment of the dosage forms of the present invention, the dosage forms comprise meclizine or salts, and/or solvates.

In other embodiments of the present invention, the plurality of beads in a dosage form can yield different desired drug (e.g., meclizine) release profiles. In one embodiment, for example, a once-daily dosage form comprising a weakly basic, piperazine derivative of an H₁-receptor antagonists with an elimination half-life of from about 2 hours to 14 hours may contain a mixture of a population of taste masked IR particles (which provides an immediate-release pulse of the weakly basic drug) and one or more CR particle populations, exhibits the target in vitro release profile over about 8-18 hours, and maintains clinically effective plasma concentrations of the drug over about 12-24 hours.

The step of preparing the core may be accomplished by any of the methods known in the art; for example, layering an organic acid onto an inert bead (e.g., sugar, microcrystalline cellulose, mannitol-microcrystalline cellulose, silicon dioxide, etc.) with a solution comprising the acid and optionally a polymeric binder (e.g., by fluid-bed or pan coating), or by controlled spheronization or powder layering using Granurex from Vector Corporation, etc.) Alternatively, “preparing a core” can comprise obtaining or preparing organic acid particles or crystals of the desired particle size (e.g., about 100-500 pin, including about 150-250 μm).

In some embodiments, the method comprises preparing core particles comprising the weakly basic, piperazine derivative of an H₁-receptor antagonist (e.g., meclizine as described herein), then coating the core particles with an SR coating (as described herein), followed by a TPR coating (as described herein) or a DR coating (as described herein). In other embodiments, the method comprises preparing core particles comprising the drug, and then coating the core particles with a TPR coating, followed by a DR coating. Each of these embodiments, optional sealant layers can be applied under, over, and/or between the controlled-release layers.

In yet another embodiment, the method of the present invention further comprises filling therapeutically effective amounts of IR beads and one or more CR bead populations comprising the weakly basic, piperazine derivative of an H₁-receptor antagonist into hard gelatin capsules. Alternatively, appropriate amounts of taste-masked IR and CR bead populations and rapidly dispersing microgranules are blended in a V blender and compressed into ODTs using a rotary tablet press equipped with an external lubrication device to lubricate die and punch surfaces prior to each compression in accordance with the disclosures of U.S. Pat. No. 5,700,492 B1 and U.S. Pat. No. 6,764,695 B1. Each of these patents set forth herein are incorporated by reference in their entireties for all purposes.

In another embodiment, the method further comprises coating a compressible layer comprising a hydrophilic polymer (e.g., hydroxypropylcellulose), over the controlled-release layers to eliminate/minimize damage to the extended-release coating(s) of the CR particles during compression into an ODT.

In yet another embodiment, the method of the present invention further comprises blending the controlled-release composition described herein with optional excipients (e.g., additional disintegrant, compression aid such as microcrystalline cellulose, a sweetener, a flavorant, a colorant), and compressing the blended composition into a tablet.

In another embodiment, the method of the present invention comprises the steps of:

(a) preparing organic acid cores (crystals, microgranules, acid layered beads, or pellets by controlled spheronization using Granurex from Vector Corporation or the like with a desired average particle size (e.g., 100-400 μm or about 150-300 μm for use in ODTs or 300-600 μm or about 350-500 μm for use in CR capsules);

(b) applying a sustained-release (SR) coating comprising a water-insoluble polymer or a timed, pulsatile release (TPR) coating comprising a water-insoluble polymer in combination with an enteric polymer at a weight ratio of from about 10:1 to 1:4, onto the acid cores at a coating weight of from about 10% to 30%, thereby forming SR or TPR beads comprising an organic acid;

(c) optionally applying a TPR coating comprising a water-insoluble polymer in combination with an enteric polymer at a weight ratio of from about 10:1 to 1:4 onto the SR acid beads from step (b);

(d) preparing IR beads by applying a piperazine derivative of an H₁-receptor antagonists s (e.g., meclizine or its salt) onto the SR or TPR acid beads from step (b) or CR beads from step (c) forming drug-layered beads that are optionally provided with a protective seal-coat;

(e) applying a SR coating comprising a water-insoluble polymer or TPR coating comprising a water-insoluble polymer in combination with an enteric polymer onto the IR beads at a coating weight of from about 10% to 30%, thereby forming SR or TPR beads;

(f) optionally applying a delayed-release (DR) coating comprising an enteric polymer onto the SR or TPR beads at a coating weight of from about 10% to 30%, thereby forming controlled-release (CR) beads; and

(g) filling required amounts of IR beads from step (d) and CR beads (SR/TPR beads from step (e) or CR beads from step (f)) into hard gelatin or HPMC capsules to produce CR Capsules containing a therapeutically effective dose of the piperazine derivative (e.g., meclizine or its salt) as IR and one or more CR bead populations. In another embodiment, the method of the present invention further comprises the steps of:

(h) preparing IR beads by drug layering directly onto inert cores (e.g., 60-80 mesh sugar spheres), providing a seal coat of Opadry Clear onto IR beads prior to applying a taste-masking coating of ethyl cellulose/Eudragit E100 at a coating weight of from about 10% to 30%, thereby forming taste-masked IR beads;

(i) preparing a plurality of rapidly-dispersing microgranules comprising a disintegrant (e.g., crospovidone or low substituted hydroxypropylcellulose) and a sugar alcohol (e.g., mannitol with a mean particle size of not more than about 30 μm) and/or a saccharide (e.g., lactose with a mean particle size of not more than about 30 μm);

(j) blending required amounts of the SR or TPR beads from step (e) and/or CR beads from step (f), taste-masked IR beads from step (h), rapidly dispersing microgranules from step (i), microcrystalline cellulose (about 10% based on tablet weight), additional disintegrant (about 5 wt. %), sucralose, mint flavor; and

(k) compressing the blend into orally disintegrating tablets on a rotary tablet press equipped with an external lubricating devise to lubricate the die and punch surfaces prior to each compression to produce ODTs that rapidly disintegrate on contact with saliva in the oral cavity into a smooth, easy-to-swallow suspension containing coated beads.

In still another embodiment, the present invention is directed to a method of use comprising the administration of a therapeutically effective amount of a composition of the present invention, (e.g. meclizine once-daily dosage forms such as Meclizine CR Capsules or Meclizine ODT CR) as an antivertigo/antiemetic agent to a patient in need thereof, in the management of nausea, vomiting, and dizziness associated with motion sickness and vertigo in diseases affecting the vestibular apparatus.

The present invention is described in greater detail in the sections below. The following examples involving meclizine, a member of the piperazine class of H1-receptor antagonists, are used to illustrate the present invention. It should be understood that the examples and embodiments described herein are for illustrative purposes only and that various modifications or changes in light thereof will be suggested to persons skilled in the art and are to be included within the spirit and purview of this application.

### EXAMPLES

### Example 1

### 1.A Fumaric Acid Layered Beads

Hydroxypropylcellulose (Klucel LF) is slowly added into 90/10 denatured SD 3 C 190 proof alcohol/water mixture while stirring rather vigorously to dissolve and then fumaric acid is added slowly. Talc is homogenized into the polymer solution, if required, to minimize static build-up. A Glatt GPCG 3 equipped with a 7″ bottom spray/8″ column height Wurster insert, 20 mm partition gap, air-distribution plate B (250 μm screen), 1.0 mm nozzle port, atomization air pressure of 2 bar, and 3.2 mm inner diameter tubing; atomization air pressure: 2 bar; product temperature: 35±2° C.), inlet air volume: ˜150 CFM; spray rate: 8 to 30 g/min) is charged with 25-30 mesh sugar spheres and coated with the fumaric acid layering solution by spraying at a rate of 8 to 30 g/min for a weight gain of 10% w/w. The acid cores are dried in the unit for 10 min to drive off residual solvent/moisture and sieved to discard doubles if any.

### 1.B Fumaric Acid SR Beads

Fumaric acid cores from step 1.A above are charged into Glatt GPCG 3 (e.g., equipped with a 7″ bottom spray Wurster 7 13/16″ column height, “C” bottom air distribution plate covered with a 200 mesh product retention screen) and coated with a solution (7% solids) of ethyl cellulose optionally plasticized (e.g., triethylcitrate, TEC at 10 wt. %) by spraying at a rate of 8 to 30 g/min for a weight gain of 10% w/w. The SR acid beads are dried in the unit for 10 min to drive off residual solvent/moisture and sieved to discard doubles if any.

### 1.C Meclizine IR Beads

A binder polymer such as hydroxypropylcellulose (Klucel LF) is slowly added to a solvent system (e.g., water, acetone, ethanol or a mixture thereof) to prepare a binder solution. The weakly basic drug (e.g., meclizine dihydrochloride monohydrate) is slowly added to the binder solution while mixing. The GPCG 3 is charged with SR acid beads from step 1.B above, which are then sprayed with the binder/drug solution. Following completion of drug layering, the drug layered beads are applied with a seal coat by spraying an aqueous solution of Opadry Clear for a weight gain of 2 wt. % to produce IR Beads. The IR beads are then dried to drive off residual solvents (including moisture), and can be sieved to discard oversized particles and fines.

### 1.D Meclizine SR Beads

The IR beads from Example 1.C above are coated in Glatt GPCG 3 with a SR coating of an optionally plasticized (e.g., triethylcitrate at 10% w/w of ethyl cellulose) water-insoluble polymer (e.g., ethyl cellulose) for a weight gain of 15 wt. %. Samples are pulled at coating levels of 5%, 7.5%, 10%, and 12.5% for potency and drug and acid release testing. The SR beads are then dried to drive off residual solvents (including moisture), and can be sieved (e.g., through 15 and 30 mesh screens) to discard oversized particles and fines.

### 1.E Meclizine TPR Beads

The IR beads from Example 1.C above are coated in Glatt GPCG 3 with a TPR coating of an optionally plasticized (e.g., triethylcitrate at 10% w/w of the coating) water-insoluble polymer (e.g., ethyl cellulose) in combination with an enteric polymer (e.g., hypromellose phthalate, HP-55) at a weight ratio of 60/30/10 for a weight gain of up to 25%. EC-10 (ethyl cellulose, Ethocel Premium 10 cps from Dow Chemicals) is slowly added to 90/10 acetone/water with continuous agitation for not less than 30 minutes, until dissolved. Then HP-55 (hypromellose phthalate, HP-55, from Shin Etsu Chemicals) and TEC (triethylcitrate) are added to the EC-10 solution until dissolved. The TPR coating solution is applied using a Glatt GPCG 3 equipped with a 6″ bottom spray/8″ column height Wurster insert, 20 mm partition gap, air-distribution plate D (200 mesh screen), 0.8 mm nozzle port, atomization air pressure of 1.0 bar, and 14 mm single-head tubing, PB 3% dedicated filter bag. The TPR coating solution is sprayed onto the IR beads at a spray rate of 10-15 mL/min, outlet flap at about 28% (air velocity: 3.4-3.8 m/s/pressure: 7-7.5 Pa), while maintaining the product temperature at about 32-34° C., and dried in the Glatt at the same temperature for 10 minutes to drive off excess residual solvent. Samples are pulled at coating levels of 10%, 15%, and 20% for testing for potency and drug/organic acid release profiles. The TPR beads are then dried to drive off residual solvents (including moisture), and sieved (e.g., through 14 and 30 mesh screens) to discard oversized particles and fines.

### 1.F Meclizine CR (DR Coating Over SR Coating) Beads

The SR beads from Example 1.D above are coated in Glatt GPCG 3 with a DR coating of an optionally plasticized (e.g., triethylcitrate at 10% w/w) enteric polymer (e.g., hypromellose phthalate, HP-55) for a weight gain of up to 15%. The TPR beads are then dried to drive off residual solvents (including moisture), and can be sieved (e.g., through 14 and 30 mesh screens) to discard oversized particles and fines.

### Example 2

### 2.A Meclizine IR Beads

The weakly basic drug (e.g., meclizine dihydrochloride monohydrate) is slowly added to the binder solution as disclosed in Ex. 1.C above. The GPCG 3 is charged with 25-30 mesh sugar spheres, which are then sprayed with the binder/drug solution. Following completion of drug layering, the drug layered beads are applied with a seal coat by spraying an aqueous solution of Opadry Clear for a weight gain of 2 wt. % to produce IR Beads. The IR beads are then dried to drive off residual solvents (including moisture), and can be sieved to discard oversized particles and fines.

### 2.B Meclizine SR Beads

The IR beads from Example 2.A above are coated in Glatt GPCG 3 with a SR coating of an optionally plasticized (e.g., triethylcitrate at 10% w/w of ethyl cellulose) water-insoluble polymer (e.g., ethyl cellulose) for a weight gain of 15 wt. %. Samples are pulled at coating levels of 5%, 7.5%, 10%, and 12.5% for potency and drug and acid release testing. The SR beads are then dried to drive off residual solvents (including moisture), and can be sieved (e.g., through 15 and 30 mesh screens) to discard oversized particles and fines.

### 2.0 Meclizine CR Beads

The SR beads from Example 2.B above are coated in Glatt GPCG 3 with a DR coating with an enteric polymer (e.g., hypromellose phthalate, HP-55 optionally plasticized with triethylcitrate at 10% w/w) for a weight gain of up to 25%. Samples are pulled at coating levels of 10%, 15%, and 20% for testing for potency and drug/organic acid release profiles. The CR beads are then dried to drive-off residual solvents (including moisture), and sieved (e.g., through 14 and 30 mesh screens) to discard oversized particles and fines.

### 2.D Taste-Masked IR Beads

IR beads comprising a weakly basic, piperazine derivative of H1-receptor antagonists (e.g., meclizine) are prepared in Glatt GPCG 3 by spraying the drug/binder solution onto microcrystalline cellulosic spheres (e.g., Cellets 200 from Glatt) as disclosed in Example 2.A above. The IR beads are taste-masked first by solvent coacervation with ethyl cellulose (Ethocel Premium Standard 100) for a coating at 5% w/w as disclosed in the co-pending U.S. patent application Ser. No. 10/827,106 (published as US 2005/0232988 A1) and further coated with an optionally plasticized ethyl cellulose (EC-10), a gastrosoluble polymeric pore-former (e.g., Eudragit EPO) at a ratio of about 1:1 for a weight gain of 20 wt. % as disclosed in the co-pending U.S. patent application Ser. No. 11/248,596 (published as US 2006/0078614 A1) and dried in the same fluidized bed coater to drive off residual solvents.

### Example 3

### 3.A TPR Coated Organic Acid Crystals

Fumaric acid crystals (50-80 mesh) are coated in Glatt GPCG 3 with a TPR coating of an optionally plasticized (e.g., triethylcitrate at 10% w/w) water-insoluble polymer (e.g., ethyl cellulose) in combination with an enteric polymer (e.g., hypromellose phthalate, HP-55) at a weight ratio of 65/25/10 for a weight gain of up to 35%. The TPR beads are then dried to drive-off residual solvents (including moisture), and can be sieved to discard oversized particles and fines

### 3.B Meclizine IR Beads

Glatt GPCG 3 is charged with TPR coated fumaric acid crystals of Ex. 3.A above, which are then sprayed with the binder/drug solution following the disclosures from step 1.C above. Following completion of drug layering, the drug layered beads are applied with a 2 wt. % Opadry Clear protective seal coat.

### 3.0 Meclizine CR Beads

IR beads from Example 3.B above are first coated in the same fluidized bed coater with a SR coating of an optionally plasticized (e.g., triethylcitrate at 10% w/w) ethyl cellulose (EC-10) for a weight gain of 20% in Glatt GPCG 3, Samples are pulled at coating levels of 10% and 15% by weight. Following completion of SR coating, the beads are further coated with a solution of EC-10/HP-55/Tec at a ratio of 65/25/10 for a weight gain of 25% w/w as disclosed in Ex. 1.E above. Following completion of coating, the beads are dried in the Glatt at the same temperature for 10 minutes to drive off excess residual solvent. The dried beads are sieved to discard any doubles if formed.

### 3.D Compressible Coated CR Beads

A compressible coating solution (e.g., hydroxypropylcellulose such as Klucel® LF) dissolved in a solvent is sprayed onto CR Beads of Ex. 3.0 above for a weight gain of about 3% by weight. The resulting compressible coated CR beads are dried in the same unit to drive-off residual solvents.

### 3.E Rapidly Dispersing Microgranules

The rapidly dispersing microgranules are prepared following the procedure disclosed in co-pending U.S. patent application Ser. No. 10/827,106 (published as US 2005/0232988 A1), and 12/166,757 (US 2009/0092672 A1), the contents each of which are hereby incorporated by reference for all purposes. D-mannitol with an average particle size of approximately 20 μm or less (e.g., Pearlitol 25 from Roquette, France) are blended with 8 kg of cross-linked povidone (e.g., Crospovidone XL-10 from ISP) in a high shear granulator (GMX 600 from Vector) and granulated with purified water and wet-milled using Comil from Quadro and tray-dried to obtain a loss on drying (LOD) of less than about 1%. The dried granules are sieved, and oversized material is milled to produce rapidly dispersing microgranules with an average particle size in the range of approximately 175-300 μm.

### 3.F Controlled-Release ODT Containing IR and CR Beads

Rapidly dispersing microgranules from step 3.E above are blended with taste-masked IR beads of step 2.D. Compressible coated CR beads from step 3.D above, and other pharmaceutical acceptable ingredients, such as flavor, sweetener (e.g., sucralose), additional crospovidone, and microcrystalline cellulose (e.g., Avicel PH101) at a ratio of rapidly dispersing microgranules to totality of coated bead populations of about 3:2 in a twin shell V-blender for a sufficient time to obtain a homogeneously distributed blend for compression. ODTs comprising 50 mg of weakly basic drug are compressed using a production scale tablet press equipped with an external lubrication system at the following conditions: —tooling: 13 mm round, flat face, radius edge; compression force: 8-12 kN; mean weight: 1000 mg; mean hardness: 20-40 N; and friability: <0.50%. The resulting ODT (50 mg dose) thus produced rapidly disintegrates in the oral cavity, creating a smooth, easy-to-swallow suspension comprising coated beads and provides an expected a drug-release profile suitable for a once-daily dosing regimen.

### Example 4

### 4.A Meclizine 2HCl.H₂O IR Beads (Nominal Drug Loading: 10 wt. %)

Kollidon VA 64 (2 parts) is slowly added to a 72.5/22.5/5 mixture of 95% ethanol/acetone/water while vigorously stirring until dissolved, and then meclizine (1 part) is slowly added until dissolved. A Glatt GPCG 3 equipped with a 7″ bottom spray/8″ column height Wurster insert, 20 mm partition gap, air-distribution plate B (250 μm screen), 1.0 mm nozzle port, atomization air pressure of 1.5 bar, and 3.2 mm inner diameter tubing, is charged with 25-30 mesh Sugar Spheres. Talc is homogenized into the meclizine/polymer solution to minimize static build-up. The solid dispersion at a desired solids content, is sprayed onto the sugar spheres at a spray rate of 8-17 g/min and outlet flap at ˜60-80% (air velocity: ˜85-115 m³/hr) while maintaining the product temperature at about 36-40° C. The resulting meclizine-layered beads are dried in the Glatt unit at 40° C. for about 45 min to minimize the residual solvent level in the product.

### 4.B Meclizine TPR Beads (Coating: Eudragit RL100/L100/TEC/Talc)

Meclizine IR beads from Ex. 4.A above having a drug load of 10% are coated by spraying a solution of Eudragit RL/Eudragit L/TEC/talc at a ratio of 45/40/10/5 in acetone/ethanol (the talc was suspended in the solution using an Ultraturrex homogenizer) at a solids content of 10%, to provide coatings of up to 20% by weight (samples are pulled at coating weights of 5%, 10%, and 15%).

The TPR coating solution is prepared by first slowly adding the Eudragit RL polymer to the solvent mixture to achieve a clear solution while vigorously stirring. Next, the Eudragit L polymer and then the plasticizer (triethylcitrate or “TEC”) are slowly added and allowed to dissolve in the solution. Talc is separately homogenized in the solvent mixture before adding to the coating solution. A Glatt GPCG 3 equipped with a 7″ bottom spray/8″ column height Wurster insert, 20 mm partition gap, air-distribution plate B (250 μm screen), 1.0 mm nozzle port, atomization air pressure of 1.5 bar, and 3.2 mm inner diameter tubing, is used to apply the coating solution onto the IR beads. The TPR coating solution is sprayed at a spray rate of 4-11 g/min, outlet flap at ˜20-30% (air velocity: ˜2.0-2.5 m/s), and at a product temperature of 35-38° C. TPR beads having coating weights of about 10% and 15% are assayed for potency and dissolution tested for drug release profiles. The coated beads are dried in the Glatt at 40° C. for 15 minutes to drive off excess residual solvents. The dried beads are sieved to discard any doubles (i.e., two or more beads adhered together by the coating solution), if formed.

### 4.0 Meclizine CR Beads (SR Coating Followed by DR Coating)

Meclizine IR beads prepared as described in Ex. 4.A above, are first coated with a solution of ethyl cellulose (Ethocel 10 Premium from Dow Chemicals) and polyethylene glycol (PEG 400) at a ratio of 65/35 for a weight gain of 10% (samples at 5% and 7% coating for testing of potency and drug release profile) to form SE beads. These SR beads at a coating of 7.5% by weight are further coated with an enteric polymer (e.g. hypromellose phthalate, HP-55 from Shin Etsu Chemicals) at coating levels of 10%, 15% and 20% by weight. CR beads having coating weights of about 10% and 15% are assayed for potency and drug release profile using USP Apparatus 2-HPLC methodology.

### Example 5

### 5.A Meclizine IR Beads (60-80 Mesh Sugar Spheres)

Meclizine IR beads (nominal drug load: 10% by weight) are prepared by spraying a 1:2 solid solution of meclizine:Kollidon VA 64 onto 60-80 mesh sugar spheres in a Glatt GPCG 3, following procedures similar to those described above in 4.A.

### 5.B Meclizine TPR Beads (Coating: Eudragit RL/Eudragit L/TEC/Talc)

The IR beads prepared as described above in Ex. 5.A, are coated by spraying a solution of Eudragit RL100/L100/TEC/talc at a ratio of 45/40/10/5 dissolved in ethanol/acetone/water (˜7.5% solids) at a coating weight of up to 30% by weight (samples are pulled at coating levels of about 10%, 15%, 20% and 25% for testing for potency and drug release profile) as disclosed in Ex. 4.0 above.

### 5.0 Taste-Masked Meclizine IR Beads

Meclizine IR beads prepared as described above in Ex. 5.A, are coated by spraying a solution of Ethocel/Eudragit E100/TEC/talc at a ratio of 40/40/10/10 for a coating weight of 10% in a Glatt GPCG 3, following procedures similar to those described above in Ex. 4.B.

### 5.D Meclizine 2HCl.H₂O IR ODT

Required quantities of meclizine TPR beads and taste-masked IR beads at a ratio 65/35 as drug), prepared as described above in Ex. 5.B and 5.D, respectively, rapidly dispersing granules, microcrystalline cellulose (MCC at 10 wt. %), crospovidone (5 wt. %), sucralose (0.35 wt. %), mint flavor (0.65 wt. %), and FD&C Red (0.15 wt. %) are blended together and compressed into 50 mg ODT tablets following procedures similar to those described above in Ex. 3.F. The tablets thus produced are found to rapidly disintegrate in the oral cavity creating a smooth, easy-to-swallow suspension containing taste-masked and TPR beads which provide therapeutically effective plasma concentration-time profiles.

### Example 6

### 6.A Meclizine IR Beads (Meclizine/VA64/Formic Acid)

Meclizine IR beads (nominal drug load: 10% by weight) are prepared by spraying a solution of meclizine/Kollidon VA 64/formic acid at a ratio of 1:2:1 onto 25-30 mesh sugar spheres in a Glatt GPCG 3, following procedures similar to those described above in 4.A.

### 6.B Meclizine CR Beads (SR Coating Followed by TPR Coating)

Meclizine IR beads prepared as described above in Ex. 6.A, are coated by spraying a 85/10/5 solution of Eudragit RL/TEC/talc at a coating weight of 10%, in a Glatt GPCG 3, following the procedures described above in Ex. 4.B, and are dried in the Glatt at 40° C. for 10 minutes to drive off excess residual solvent to form SR beads. The SR beads are further coated with Eudragit RL/Eudragit L/TEC/talc at a ratio of 40/45/10/5 for a weight gain of 15%. The dried CR beads are sieved to discard any doubles, if formed. CR beads having coating weights of 7.5% and 10% are assayed for potency and drug release profile using HPLC methodology.

The skilled artisan will recognize that the above procedures and compositions can be suitably modified to provide the appropriate dose of the weakly basic, piperazine derivative.

While the invention has been described in connection with the specific embodiments herein, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A pharmaceutical multiparticulate composition comprising a plurality of drug particles comprising a weakly basic, piperazine derivative of H₁-receptor antagonists, each particle comprising:
a) an organic acid core comprising a pharmaceutically acceptable organic acid;
b) a first coating disposed over the organic acid cores comprising at least one water-insoluble polymer, thereby forming a controlled release coated acid core;
c) a second coating disposed over said controlled release coated acid core comprising a weakly basic piperazine derivative of H₁-receptor antagonists and a polymeric binder; and
d) a third coating disposed over the drug core comprising at least one water-insoluble polymer, thereby forming a controlled release coated drug particle.

2. The pharmaceutical multiparticulate composition of claim 1, wherein said organic acid core comprises a pharmaceutically acceptable organic acid crystal, or a coating layer comprising an organic acid and a polymeric binder, disposed over an inert core selected from the group consisting of a sugar sphere, cellulosic sphere, cellulose-lactose, cellulose-mannitol, or fused silicon dioxide sphere.

3. (canceled)

4. A pharmaceutical multiparticulate composition comprising a plurality of drug particles comprising a weakly basic piperazine derivative of H₁-receptor antagonists, each particle comprising:
a) a core comprising a solid dispersion of said weakly basic piperazine derivative of H₁-receptor antagonists in a pharmaceutically acceptable solubility-enhancing water soluble polymer;
b) a first coating disposed over said solid dispersion core comprising at least one water-insoluble polymer, thereby forming a controlled release coated drug particle.

5. The pharmaceutical multiparticulate composition of claim 1 or 4, wherein said weakly basic piperazine derivative of H₁-receptor antagonists is selected from the group consisting of buclizine, cinnarizine, cyclizine, hydroxyzine, meclizine, and niaprazine.

6. The pharmaceutical multiparticulate composition of claim 1 or 4, wherein said weakly basic piperazine derivative of H₁-receptor antagonists is meclizine or a pharmaceutically acceptable salt or solvate thereof.

7. The pharmaceutical multiparticulate composition of claim 1 or 4, wherein said first coating further comprises a water-soluble polymer wherein the ratio of the water-insoluble polymer to the water-soluble polymer is about 85:15 to about 50:50.

8. (canceled)

9. The pharmaceutical multiparticulate composition of claim 1, wherein said third coating further comprises a water-soluble polymer wherein the ratio of the water-insoluble polymer to the water-soluble polymer is about 85:15 to about 50:50.

10. (canceled)

11. The pharmaceutical multiparticulate composition of claim 1 further comprising (e) a fourth coating disposed over the third coating comprising at least one enteric polymer.

12. The pharmaceutical multiparticulate composition of claim 11, wherein said third and fourth coatings are applied in either order.

13. The pharmaceutical multiparticulate composition of claim 4 further comprising (c) a second coating disposed over the first coating comprising at least one enteric polymer.

14. The pharmaceutical multiparticulate composition of claim 13, wherein said first and second coatings are applied in either order.

15. The pharmaceutical multiparticulate composition of claim 1 or 4, wherein at least one of the coating layers further comprises a plasticizer selected from the group consisting of polyethylene glycol, triacetin, triethyl citrate, tributyl citrate, acetyl tri-n-butyl citrate, diethyl phthalate, dibutyl phthalate, dibutyl sebacate, monoacetylated and diacetylated glycerides (e.g., Myvacet® 9-45), and mixtures thereof.

16. The pharmaceutical multiparticulate composition of claim 1 or 2, wherein said polymeric binder is selected from the group consisting of hydroxypropyl cellulose, hydroxypropyl methylcellulose, polyvinylpyrrolidone and mixtures thereof.

17. The pharmaceutical multiparticulate composition according to claim 1 or 4, wherein the water-insoluble polymer is selected from the group consisting of ethyl cellulose, cellulose acetate, cellulose acetate butyrate, polyvinyl acetate, neutral methacrylic acid/methylmethacrylate copolymers, and mixtures thereof.

18. The pharmaceutical multiparticulate composition of claim 11 or 13, wherein the enteric polymer is selected from the group consisting of cellulose acetate phthalate, hydroxypropyl methylcellulose phthalate, hydroxypropyl methylcellulose acetate succinate, polyvinyl acetate phthalate, pH-sensitive methacrylic acid/methylmethacrylate copolymers, shellac, and mixtures thereof.

19. The pharmaceutical multiparticulate composition of claim 1, 4, 11, or 13, wherein the water-insoluble polymer is ethyl cellulose and the enteric polymer is hydroxypropylmethyl cellulose phthalate.

20. The pharmaceutical multiparticulate composition of claim 1, 4, 11, or 13, wherein the water-insoluble polymer is Eudragit RL polymer and the enteric polymer is Eudragit L polymer.

21. The pharmaceutical multiparticulate composition of claim 1, wherein the weight of the second coating comprising said weakly basic piperazine derivative is from about 5 to about 40 wt % of the total weight of the drug particles and wherein the ratio of said weakly basic piperazine derivative and organic acid varies from about 5:1 to about 1:5.

22. (canceled)

23. The pharmaceutical multiparticulate composition of claim 4, wherein the weight of said solid solution coating ranges from about 5 to about 40 wt % relative to the total weight of the drug particles and wherein the ratio of said weakly basic piperazine derivative and solubility-enhancing/crystallization-inhibiting polymer varies from about 5:1 to about 1:5.

24. The pharmaceutical multiparticulate composition of claim 1, wherein the organic acid is selected from the group consisting of citric acid, fumaric acid, malic acid, maleic acid, tartaric acid, succinic acid, oxalic acid, aspartic acid, glutamic acid and mixtures thereof.

25. The pharmaceutical multiparticulate composition of claim 4, wherein the solubility-enhancing water-soluble polymer is selected from the group consisting of Kollidon VA 64 (vinylpyrrolidone-vinyl acetate copolymer), hydroxypropyl cellulose, hydroxypropyl methylcellulose, polyvinylpyrrolidone and mixtures thereof.

26. The pharmaceutical multiparticulate composition of claim 1 or 4, which further comprises immediate release drug particles comprising said weakly basic piperazine derivative of H₁-receptor antagonists.

27. (canceled)

28. The pharmaceutical multiparticulate composition of claim 26, wherein the immediate release particles further comprise a taste-masking coating comprising a water-insoluble polymer alone, or in combination with a gastrosoluble pore-former at a ratio of from about 9:1 to about 5:5, wherein the taste-masking coating ranges from about 5% to about 40 wt. % of the total weight of the taste-masked particles.

29. (canceled)

30. The composition of claim 1 or 4 further comprising a plurality of rapidly-dispersing microgranules each having an average particle size of not more than about 400 μm and comprising (i) a disintegrant and (ii) a sugar alcohol and/or a saccharide, wherein said sugar alcohol and/or saccharide each having an average particle size of not more than about 30 μm wherein the ratio of rapidly-dispersing microgranules to drug particles ranges from about 6:1 to about 2:1.

31. (canceled)

32. The composition of claim 30, wherein said rapidly-dispersing microgranules comprise:
(a) said sugar alcohol selected from the group consisting of mannitol, xylitol, maltitol, isomalt, lactitol, and sorbitol,
(b) said saccharide selected from the group consisting of sucrose, lactose, maltose, and combinations thereof; and
(c) said disintegrant selected from the group consisting of crosslinked polyvinylpyrrolidone, sodium starch glycolate, crosslinked carboxymethylcellulose of sodium, low-substituted hydroxypropylcellulose and mixtures thereof.

33. (canceled)

34. A dosage form comprising a therapeutically effective amount of the multiparticulate composition of claim 1 or 4, for administration in a patient in need thereof for treating nausea, vomiting, and dizziness associated with motion sickness and vertigo in diseases affecting the vestibular apparatus is a controlled-release capsule or tablet, wherein said controlled-release capsule or tablet exhibits target in vitro drug-release/in vivo plasma concentration profile suitable for a once- or twice-daily dosing regimen in patients in need thereof.

35. (canceled)

36. An orally disintegrating tablet (ODT) comprising the composition of claim 30, wherein said orally disintegrating tablet exhibits the following properties:
(a) a friability of less than 1% by weight; and
(b) a disintegration time of about 60 seconds or less on contact with the saliva in the oral cavity,
whereby said disintegrated orally disintegrating tablet forms a smooth (non-gritty), easy-to-swallow suspension of drug-containing microparticles in the oral cavity.

37. A method of preparing the controlled release composition of claim 1, comprising:
(a) preparing a plurality of acid cores comprising a pharmaceutically acceptable organic acid and optionally a polymeric binder;
(b) coating said acid cores with a first coating comprising water insoluble polymer and an optional water soluble polymer enteric polymer, thereby producing coated organic acid cores;
(c) coating said coated organic acid cores with a second coating comprising a weakly basic piperazine derivative of H₁-receptor antagonists or a pharmaceutically acceptable salt or solvate thereof, thereby producing drug cores;
(d) coating said drug cores with a third coating comprising a water insoluble polymer; and
(e) optionally coating said coated drug cores with said fourth coating comprising an enteric polymer and/or a water insoluble polymer
(f) if needed, taste-masking immediate release drug particles with a water-insoluble polymer alone or in combination with a gastrosoluble pore former; and
(g) combining an immediate-release particle population together with at least one controlled-release drug particle population in capsules, or compressed with rapidly dispersing microgranules into orally disintegrating tablets,
wherein said orally disintegrating tablets rapidly disintegrate on contact with saliva in the oral cavity of a patient in need thereof; and
said orally disintegrating tablets meet the disintegration time specification of not more than 30 seconds when tested in accordance with the United States Pharmacopoeia method (<701>) for disintegration time.

38. A method of preparing the multiparticulate composition of claim 4, comprising:
(a) preparing a plurality of solid solution particles comprising coating a solution of a pharmaceutically acceptable crystallization-inhibiting polymer and a weakly basic piperazine derivative of H₁-receptor antagonists or pharmaceutically acceptable salt or solvate thereof onto inert cores, thereby forming a coating of a solid solution of the weakly basic piperazine derivative of H₁-receptor antagonists and crystallization-inhibiting polymer on the inert cores;
(b) optionally coating said solid solution particles with a protective sealant coating;
(c) coating said solid solution particles with a first coating comprising a water insoluble polymer and an optional water soluble or enteric polymer; and
(d) optionally coating said coated solid solution particles with a second coating comprising an enteric polymer and an optional water insoluble polymer
(e) if needed, taste-masking immediate-release drug particles with a water-insoluble polymer alone or in combination of a gastrosoluble pore former; and
(f) combining an immediate-release particle population together with at least one controlled-release drug particle population in capsules, or compressed with rapidly dispersing microgranules into orally disintegrating tablets,
wherein said controlled-release capsules or tablets exhibit desired in vitro drug-release/in vivo plasma concentration profiles suitable for a once- or twice-daily dosing regimen in patients in need thereof.

39. (canceled)
